(19) United States
(12) Patent Application Publication
(10) Pub. No.: US 2024/0363025 A1
(43) Pub. Date: Oct. 31, 2024

(54) Simulation Device for Capillary Blood Collection

(71) Applicant: Becton, Dickinson and Company, Franklin Lakes, NJ (US)

(72) Inventors: Anthony V. Torris, Mercer Island, WA (US); Vlad Yakhnich, Park Ridge, NJ (US); Charles Peter Althoff, West Nyack, NY (US)

(21) Appl. No.: 18/574,821

(22) PCT Filed: Jun. 23, 2022
(86) PCT No.: PCT/US22/34634

§ 371 Date: Dec. 28, 2023

Related U.S. Application Data
(60) Provisional application No. 63/216,264, filed on Jun. 29, 2021.

(51) Int. Cl. G09B 23/28

(52) U.S. Cl. CPC G09B 23/285

(57) ABSTRACT

A simulation device allowing a user to practice use of a blood collection device includes a base support; a puncture target connected to the base support configured to be repeatedly punctured by a lancet of the blood collection device to practice use of the capillary blood collection device; and a fluid circulating system. The fluid circulating system includes a fluid reservoir, at least one port positioned within the puncture target, tubing extending at least partially through the puncture target between the reservoir and the at least one puncture port, and a pump for circulating the fluid from the reservoir to the at least one puncture port. The puncture target is sized to be connected to the blood collection device so that the capillary blood collection device punctures the target and the at least one puncture port to establish fluid communication with the tubing and reservoir.

### CROSS-REFERENCE TO RELATED APPLICATIONS

This application is the United States national phase of International Application No. PCT/US2022/034634 filed Jun. 23, 2022, and claims priority to U.S. Provisional Application Ser. No. 63/216,264, filed Jun. 29, 2021, entitled “Simulation Device for Capillary Blood Collection”, the entire disclosures of which are hereby incorporated by reference in their entireties

### BACKGROUND OF THE INVENTION

### Field of the Invention

The present disclosure relates generally to devices, training systems, and methods that allow users to practice various tasks related to obtaining a capillary blood sample from a patient using a capillary blood collection device. More particularly, the present disclosure relates to a simulation device that allows the user to practice sizing and use of finger-based capillary blood collection devices, such as devices configured to lance and squeeze a finger, collect, stabilize, and dispense a blood sample in a controlled manner.

### Description of Related Art

Devices for obtaining and collecting biological samples, such as blood samples, are commonly used in the medical industry. One type of blood collection that is commonly done in the medical field is capillary blood collection, which is often done to collect blood samples for testing. Certain diseases, such as diabetes, require that a patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member, such as a needle, or a sharp-edged member, such as a blade, that is used to make a quick puncture wound or incision in the patient's skin in order to provide a small outflow of blood. In order to simplify capillary blood collection, lancet devices have evolved into automatic devices that puncture or cut the skin of the patient upon actuation of a triggering mechanism. In some devices, the needle or blade is kept in a standby position until it is triggered by the user. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releasing the lever from interference engagement with the lancet structure. The blood sample that is received is then collected and/or tested. This testing can be done by a Point-of-Care (POC) testing device or it can be collected and sent to a testing facility.

Use of lancet devices for capillary blood collection can be complex requiring a high skill level for the healthcare worker performing the blood collection procedure. The multi-step nature of the capillary blood collection process can introduce several variables that may cause sample quality issues, such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

While capillary blood collection devices and assemblies have been developed to simplify the capillary blood collection processes, such as finger-based capillary blood collection devices configured to lance and squeeze a finger, collect, stabilize, and dispense a blood sample in a controlled manner, it is still necessary that healthcare workers practice capillary blood collection processes to gain experience and reduce error. The simulation devices, training systems, and methods disclosed herein are intended to provide opportunities for healthcare workers to gain such experience prior to interacting with patients.

### SUMMARY OF THE INVENTION

According to an aspect of the disclosure, a simulation device allowing a user to practice use of a capillary blood collection device includes a base support; a puncture target connected to the base support configured to be repeatedly punctured by a lancet of the capillary blood collection device to practice use of the capillary blood collection device; and a fluid circulating system. The fluid circulating system includes a fluid reservoir, at least one port positioned within the puncture target, tubing extending at least partially through the puncture target between the reservoir and the at least one puncture port, and a pump for circulating the fluid from the reservoir to the at least one puncture port. The puncture target is sized to be connected to the capillary blood collection device so that the capillary blood collection device punctures the target and the at least one puncture port to establish fluid communication with the tubing and reservoir. When the fluid communication is established and the pump is circulating the fluid from the reservoir, fluid is expelled from the at least one port and is collected within a container of the blood collection device.

According to another aspect of the disclosure, a method for practicing sizing and use of a capillary blood collection device includes a step of inserting an elongated member of the puncture target of the previously described simulation device through one or more openings in a sizing card to identify an opening that best fits the elongated member of the puncture target. The method further includes steps of inserting a holder of a capillary blood collection device of a size corresponding to the identified openings onto the elongated member of the puncture target, and activating the capillary blood collection device to puncture the elongated member and the at least one puncture portion of the capillary blood device. Finally, the method includes a step of activating the pump of the fluid circulation system, thereby allowed circulating fluid to collect in a container of the capillary blood collection device.

Non-limiting illustrative examples of embodiments of the present disclosure will now be described in the following numbered clauses:
Clause 1: A simulation device allowing a user to practice use of a capillary blood collection device, the simulation device comprising: a base support; a puncture target connected to the base support configured to be repeatedly punctured by a lancet of the capillary blood collection device to practice use of the capillary blood collection device; and a fluid circulating system comprising a fluid reservoir, at least one port positioned within the puncture target, tubing extending at least partially through the puncture target between the reservoir and the at least one puncture port, and a pump for circulating the fluid from the reservoir to the at least one puncture port, wherein the puncture target is sized to be connected to the capillary blood collection device so that the capillary blood collection device punctures the target and the at least one puncture port to establish fluid communication with the tubing and reservoir, and wherein, when the fluid communication is established and the pump is circulating the fluid from the reservoir, fluid is expelled from the at least one port and is collected within a container of the capillary blood collection device.
Clause 2: The simulation device of clause 1, wherein the base support is configured to support the puncture target with at least 6.0 inches of vertical clearance between the puncture target and a bottom of the base support.
Clause 3: The simulation device of clause 1 or clause 2, wherein the base support comprises a first member configured to rest on a surface and a second member extending from the first member, wherein the puncture target extends from the second member of the base support.
Clause 4: The simulation device of any of clauses 1-3, wherein the base support comprises a flexible arm rotatably and/or pivotally mounted to a support member.
Clause 5: The simulation device of clause 4, wherein the flexible arm comprises at least one hinge configured to bend in one or multiple directions to adjust a position of the puncture target relative to the support member.
Clause 6: The simulation device of clause 4 or clause 5, wherein the flexible arm pivots and/or rotates to raise or lower a height of the puncture target relative to a bottom of the base support.
Clause 7: The simulation device of any of clauses 1-6, wherein the pump comprises an electric pump.
Clause 8: The simulation device of any of clauses 1-6, wherein the pump comprises a manually operated mechanical pump.
Clause 9: The simulation device of any of clauses 1-7, wherein the fluid circulating system further comprises a remote control operatively connected to the pump configured to operate and/or control a flow rate of the pump.
Clause 10: The simulation device of any of clauses 1-7, wherein the pump draws fluid from the reservoir at a flow rate of about 5 μL/s.
Clause 11: The simulation device of any of clauses 1-10, wherein the fluid in the reservoir comprises fake blood liquid.
Clause 12: The simulation device of any of clauses 1-11, wherein the puncture target comprises a substantially anatomically accurate hand model comprising a proximal end portion connected to the base support and a distal end portion comprising a plurality of elongated members representing fingers, and wherein the fluid circulating assembly comprises a plurality of ports positioned in the elongated members representing the fingers.
Clause 13: The simulation device of clause 12, wherein dimensions of the hand model are selected based on 50^{th} percentile anatomical measurements for hand size for an adult.
Clause 14: The simulation device of clause 12 or clause 13, wherein the plurality of elongated members of the hand model comprise at least a ring finger member sized to receive a first size of a capillary blood collection device and a middle finger member sized to receive a second size of capillary blood collection device that is larger than the first size.
Clause 15: The simulation device of any of clauses 12-14, wherein the hand model is oriented in a left-hand orientation, and wherein the hand model is positioned with a flat downwardly facing palm and with the plurality elongated member representing fingers spaced apart.
Clause 16: The simulation device of any of clauses 12-15, wherein the hand model comprises an internal frame that mimics bones of a human hand and an elastomeric cover over the internal frame configured to mimic a look and feel of human skin.
Clause 17: The simulation device of clause 16, wherein the cover comprises an elastomeric sheet.
Clause 18: The simulation device of any of clauses 12-17, wherein a first of the plurality of ports is positioned in an elongated member of the plurality of elongated members that represents a middle finger, and a second of the plurality of ports is positioned in an elongated member representative of a ring finger.
Clause 19: The simulation device of clause 18, wherein elongated members representative of fingers other than the middle finger or the ring finger are bendable, capable of being bent away from the middle finger and/or the ring finger thereby allowing the user to access and manipulate the middle and/or ring fingers to practice the procedure.
Clause 20: A method for practicing sizing and use of a capillary blood collection device, the method comprising: inserting an elongated member of the puncture target of the simulation device of any of clauses 1-19 through one or more openings in a sizing card to identify an opening that best fits the elongated member of the puncture target; inserting a holder of a capillary blood collection device of a size corresponding to the identified openings onto the elongated member of the puncture target; activating the capillary blood collection device to puncture the elongated member and the at least one puncture portion of the capillary blood device; and activating the pump of the fluid circulation system, thereby allowed circulating fluid to collect in a container of the capillary blood collection device.

### BRIEF DESCRIPTION OF THE DRAWINGS

FIG. 1A is a perspective view of a capillary blood collection device for obtaining a blood sample from a patient's finger and a collection container according to an aspect of the disclosure.

FIG. 1B is a cross-sectional view of the capillary blood collection device of FIG. 1A and a lancet according to an aspect of the disclosure.

FIG. 1C is a perspective view of a holder of a capillary blood collection device according to an aspect of the present disclosure.

FIG. 1D is a schematic drawing showing a top view of the holder of FIG. 1C connected to a patient's finger for performing a blood collection procedure.

FIG. 1E is another schematic drawing showing a front view of the holder of FIG. 1C connected to the patient's finger.

FIG. 2A is a perspective view showing a set of capillary blood collection devices of different sizes that can be used for capillary blood collection for a patient according to an aspect of the disclosure;

FIG. 2B is a drawing of a sizing card for determining sizing for a finger to be punctured using one of the capillary blood collection devices shown in FIG. 2A according to an aspect of the disclosure.

FIG. 3 is a perspective view of a simulation device for practicing performance of a capillary blood collection procedure according to an aspect of the disclosure;

FIG. 4 is a perspective view of another example of a simulation device for practicing performance of a capillary blood collection procedure according to an aspect of the disclosure.

FIG. 5 is a schematic drawing of a simulation device for practicing performance of a capillary blood collection procedure according to an aspect of the disclosure.

FIG. 6 is a flow chart showing a method for using a simulation device to practice performance of a capillary blood collection procedure according to an aspect of the disclosure.

FIGS. 7A-7I are drawings showing use of a simulation device for collecting a capillary blood sample using a capillary blood collection device according to aspects of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

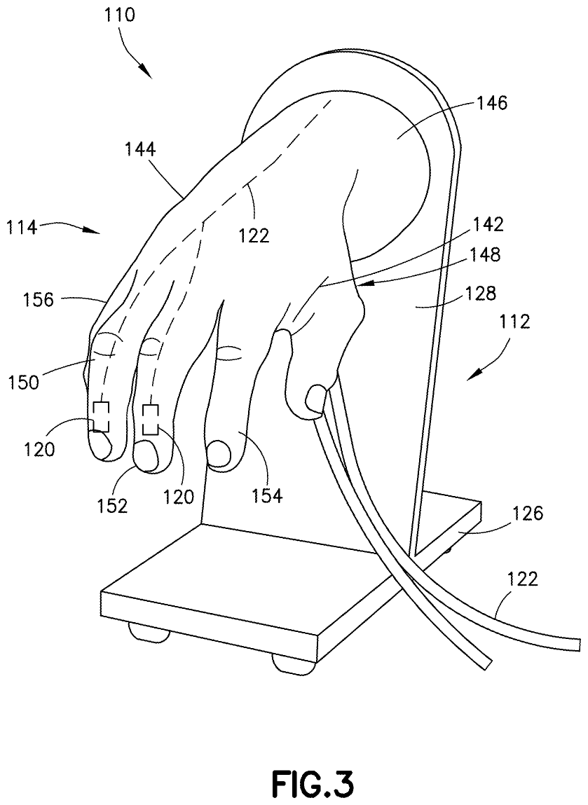

The present disclosure relates to a simulation device 110 (shown in FIGS. 3-5 and 7A-7I) that allows a user to simulate a capillary puncture procedure so that the user can become familiar with how such procedures are performed. The user can be, for example, a trained healthcare worker, such as a nurse, phlebotomists, or another trained healthcare provider, who needs to become familiar with the use of a particular capillary blood collection device 10. The user can also be a healthcare worker without blood collection experience, but who can perform minor blood collection procedures, such as a pharmacist or pharmacy technician. In some cases, the user can also be a patient who needs to gain experience using the capillary blood collection device 10 before using the device 10 to perform a puncture procedure on his or her own finger. The simulation device 110 of the present disclosure allows the user to practice steps for sizing a finger to identify a capillary blood collection device 10 of a correct size and positioning a cuff or holder 12 of the capillary blood collection device 10 at an appropriate location on a patient's finger. Once the capillary blood collection device 10 is attached to the finger, the user can practice puncturing the finger and can watch as fluid (i.e., fake blood liquid) is collected within a container 16 of the capillary blood collection device 10. In some examples, the simulation device 110 can be used for testing a healthcare worker to confirm that user/healthcare worker is comfortable using a particular type of capillary blood collection device 10. Testing can be carried out by a proctor, preceptor, or trainer. For example, the trainer can set up and operate the simulation device 110 and can observe as the user performs a simulated blood collection procedure using the simulation device 110.

In some examples, the simulation device 110 can include an anatomically accurate model of a human hand that can have a look and feel of a real hand, thereby providing a simulation experience that is as realistic as possible for the user. For example, a hand model of the simulation device 110 can include a substructure formed from plastic and metal parts (i.e., 3D printed parts) connected together to mimic bone structure of the human hand. The hand model can also include a cover formed from a rubber-like or leather material to approximate a look and feel of human skin. Fingers of the model hand can be bendable so that fingers that are not being used for the simulated blood collection procedure can be articulated or moved out of the way so that the user can position the capillary blood collection device 10 at an appropriate location on the hand model. In some examples, the hand model can be mounted to a support structure, such as a plate, that can move up and down and rotate around an axis to mimic pronation and supination of a human wrist. The hand model can also be mounted to a flexible arm or hinged arm, such as an arm used to support a computer display, for increased adjustability.

The simulation device 110 of the present disclosure can also include a fluid circulation system including, for example, flexible or rigid tubing configured to carry fluid (i.e., fake blood liquid) from a reservoir through the hand model and a pump for controlling fluid flow through the fluid circulation system. The user (or a trainer or proctor) can operate the pump by, for example, starting or stopping the pump at appropriate times and/or controlling a flow rate of the pump. Circulating fluid is expelled from the tubing or other portions of the fluid circulation system 110 into a collection container of the capillary blood collection device 10 to simulate a blood draw procedure.

### Capillary Blood Collection Device

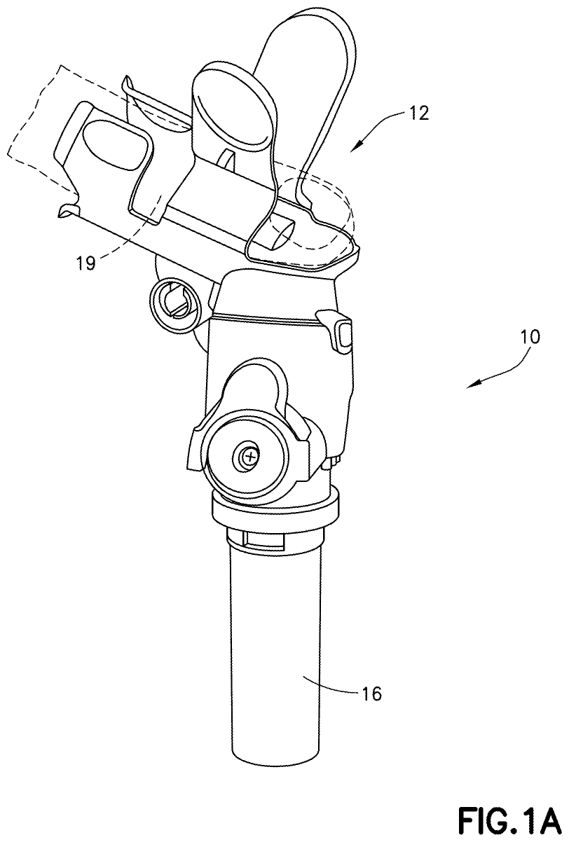

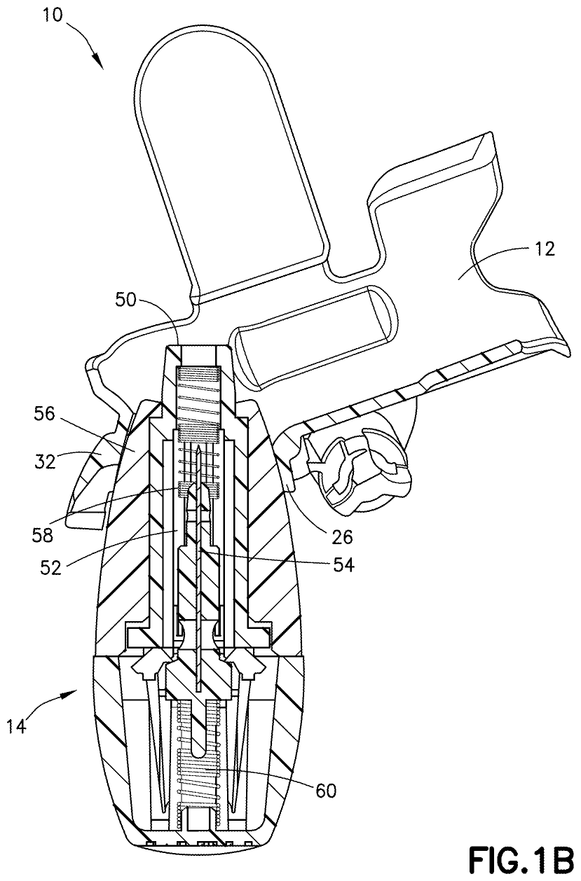

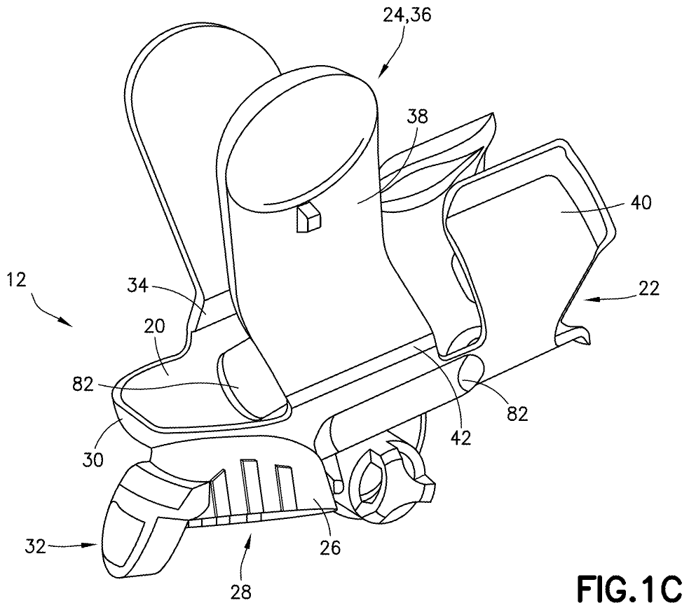

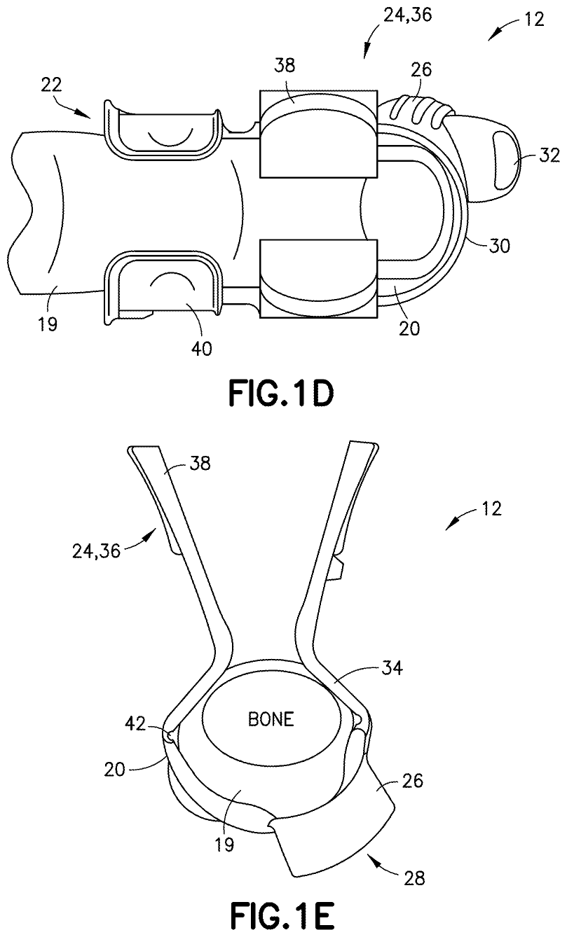

As previously described, the simulation device 110 of the present disclosure allows the user to practice performing a puncture procedure using a capillary blood collection device 10. Examples of capillary blood collection devices 10 that can be used with the simulation device 110 of the present disclosure are shown in FIGS. 1A-1E. The capillary blood collection devices 10 can be, for example, a self-contained and fully integrated finger-based capillary blood collection device with the ability to lance, collect, and stabilize a high volume capillary blood sample, e.g., up to or above 500 microliters. The simulation device 110 can also be used with other types of blood collection devices 10, such as devices that are formed from separable components (i.e., a finger cuff, lance, and reservoir) that can be connected and/or used together to obtain a blood sample. The simulation device 110 can also be used to train healthcare workers to use other types of puncture, lancet, and blood collection devices, as well as other venous access devices, such as syringes, needle cannulas, and other injection devices, as are known in the medical device art. Other exemplary capillary blood collection devices and assemblies that can be used with the simulation device 110 of the present disclosure are described, for example, in United States Patent Appl. Pub. No. 2019/0216380, entitled “Device for Obtaining a Blood Sample”; United States Patent Appl. Pub. No. 2019/0223772, entitled “Device for the Attached Flow of Blood”; and PCT Publication No. WO 2020/167746, entitled “Capillary collector with rotatable connection,” each of which is incorporated herein by reference in its entirety.

With reference to FIGS. 1A and 1B, the example capillary blood collection device 10 includes an integrated holder 12, a lancet housing or lancet 14 (shown in FIG. 1B) for puncturing a finger 19 (shown in FIGS. 1D and 1E) of the patient, and a collection container 16 (shown in FIG. 1A). In other examples, the capillary blood collection device 10 can be provided as a semi-integrated device 10 including, for example, an integrated lancet housing and collection container that can be connected with a separate holder. In other examples, a semi-integrated device may have an in-line flow and an integrated lancet housing and collection container, which can be connected with a separate holder.

The holder 12 is configured to receive a sample source, e.g., the finger 19 of a patient, for supplying a biological sample, such as a blood sample. As shown in FIGS. 1C-1E, the holder 12 generally includes a finger receiving portion 20 having a first opening 22, an actuation portion 24, a port 26 having a second opening 28, and a finger end guard 30. In some examples, the finger end guard 30 provides a stop portion for properly aligning and securing the finger 19 within the holder 12. The finger end guard 30 further assists in ensuring the patient's finger 19 is placed at a proper position within the finger receiving portion 20 so that applied pressure to the patient's finger 19 will result in adequate blood flow. The finger end guard 30 can have a curved fingertip rest that ensures the patient's finger 19 stops at an end of the finger receiving portion 20, while permitting the patient's finger nail to clear the end of the finger receiving portion 20. The finger receiving portion 20 permits use of the holder 12 with artificial and natural fingernail styles present in the patient population.

The first opening 22 of the finger receiving portion 20 is configured for receiving the sample source, e.g., the finger 19. The sample source may also include other parts of the body capable of fitting within the first opening 22, such as toes or other extremities. The port 26 is in communication with the finger receiving portion 20. For example, with a finger 19 received within the holder 12, the port 26 is in communication with a portion of the finger 19. As described in further detail herein, the holder 12 can be sized for use with a particular subset of patients. For example, a small holder may be an appropriate size of a bottom quartile of patients. Medium and large-sized holders 12 can be sized for use with patients in the lower middle quartile of patients (25% to 50%) and the upper middle quartile of patients (50% to 75%), respectively.

The second opening 28 of the port 26 is configured for receiving the lancet housing or lancet 14 (shown in FIG. 1B) and a collection container 16 (shown in FIG. 1A). In some examples, the port 26 further includes a locking portion 32 for securely receiving the lancet housing or lancet 14 and the collection container 16 within the port 26.

The actuation portion 24 of the device 10 is transitionable between a first position, in which the holder 12 defines a first diameter, and a second position, in which the holder 12 defines a second diameter, with the second diameter being less than the first diameter. Further, in the first position, the holder 12 defines a first elliptical shape. In the second position, the holder 12 defines a second elliptical shape, with the first elliptical shape being different than the second elliptical shape. In this manner, with the holder 12 in the second position with a reduced diameter, a portion of the holder 12 contacts the sample source (i.e., the finger 19) and the actuation portion 24 of the holder 12 is able to pump and/or extract blood, as described in more detail below.

In some examples, the actuation portion 24 includes a contact member 34. With the actuation portion 24 in the first position, the contact member 34 is in a disengaged position, i.e., the contact member 34 is provided in a first position with respect to the sample source, such that the contact member 34 may be in slight contact therewith. With the actuation portion 24 in the second position, the contact member 34 is in an engaged position, i.e., the contact member 34 is provided in a second position with respect to the finger 19, such that the contact member 34 is in an applied pressure contact with the finger 19, and the actuation portion 24 of the holder 12 is able to pump and/or extract blood. For example, with the contact member 34 in the engaged position, the contact member 34 exerts a pressure on the sample source.

In some examples, the actuation portion 24 includes a pumping member 36 for applying pressure to the finger 19, such as a pair of opposed tabs or wings 38. Each wing 38 can include a contact member 34. The holder 12 can also include a living hinge portion 42. The living hinge portion 42 allows the user to squeeze the wings 38 between a first position (passive state) and a second position (active state). It is believed that use of the tabs or wings 38 to draw blood out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12, while flexing to accommodate the largest patient fingers within a holder 12 without blood occlusion. In some examples, the wings 38 may be positioned on the finger receiving portion 20 at a position located proximal of a patient's fingernail and distal of a patient's first knuckle to avoid hard tissues on the patient's finger 19.

The holder 12 can be configured to allow a user to repeatedly squeeze and release the wings 38 to pump and/or extract blood from a finger 19 until a desired amount of blood is filled in a collection container 16. The wings 38 are configured to flex to maintain gentle contact with a range of patient finger sizes that may be used with the holder 12 and to retain the holder 12 on the patient's finger 19. The wings 38 may also provide active pressure features for the holder 12.

In some examples, the holder 12 can include a stability extension portion 40. This provides additional support for the holder 12 to be securely placed onto the finger 19. In one example, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

The capillary blood collection device 10 for obtaining the blood sample also includes the lancet housing or lancet 14 (shown in FIG. 1B) that is removably connectable to the port 26 of the holder 12. Referring to FIG. 1B, the lancet housing or lancet 14 can include an inlet or opening 50, an interior 52, a puncturing element 54, an engagement portion 56, a retractable mechanism 58, and a drive spring 60. The puncturing element 54 can be moveable between a pre-actuated position, wherein the puncturing element 54 is retained within the interior 52 of the lancet housing 14, and a puncturing position, wherein at least a portion of the puncturing element 54 extends through the inlet 50 of the lancet housing or lancet 14 to lance a portion of a finger 19. In one example, the lancet 14 of the present disclosure is a contact activated lancet and may be constructed in accordance with the features disclosed in U.S. Pat. No. 9,380,975, entitled “Contact Activated Lancet Device”, which is incorporated herein by reference in its entirety.

In some examples, the holder 12 and the lancet housing or lancet 14 are separate components that can be removably connectable to the port 26 of the holder 12. In such examples, the lancet housing or lancet 14 includes the engagement portion 56. The lancet housing or lancet 14 can be pushed into the port 26 of the holder 12, such that the engagement portion 56 of the lancet housing or lancet 14 is locked within the locking portion 32 of the holder 12. In this manner, the lancet housing 14 is securely connected and locked to the holder 12, such that the puncturing element 54 of the lancet housing 14 can be activated to lance or puncture a sample source, e.g., the finger 19. In some examples, the port 26 of the holder 12 includes a plurality of ribs for securing and locking the lancet 14 or the collection container 16 in the port 26.

To activate the lancet 14, the lancet 14 is pushed against the finger 19 to activate the retractable mechanism 58 and drive spring 60 of the lancet 14 to lance the finger 19. After puncturing, the puncturing element 54 is immediately retracted and safely secured within the interior 52 of the lancet housing 14. Once the finger 19 is punctured, the blood sample is squeezed from the finger 19 into a collection container 16. The collection container 16 may also contain a sample stabilizer, e.g., an anticoagulant, to stabilize a blood sample and/or a component of a blood sample disposed therein. The collection container 16 may also include at least one fill line(s) corresponding to a predetermined volume of sample. The collection container 16 may also indicate/meter a collected volume of blood.

In order to use the capillary blood collection device 10 shown in FIGS. 1A-1E, a desired finger 19 is first cleaned and a holder 12 having an appropriate size for the desired finger 19 is selected and placed onto the finger 19 securely. Next, the lancet housing or lancet 14 is connected to the port 26 of the holder 12. As discussed previously, the lancet housing or lancet 14 is pushed into the port 26 of the holder 12, such that the engagement portion 56 of the lancet housing or lancet 14 is locked within the locking portion 32 of the holder 12. In this manner, the lancet housing or lancet 14 is securely connected and locked to the holder 12, such that the puncturing element 54 of the lancet housing 14 can be activated to lance or puncture the finger 19. With the lancet 14 connected to the port 26 of the holder 12, the lancet 14 is in communication with the finger 19.

When it is desired to activate the lancet 14 to lance the skin of the finger 19, the lancet 14 is pushed against a finger 19 to activate a retractable mechanism 58 of the lancet 14 to lance the finger 19. After the finger 19 is lanced to create blood flow from the finger 19, the lancet 14 is removed from the holder 12 and the collection container 16 is pushed into the port 26 of the holder 12. With the container 16 properly secured to the holder 12 for collection of a blood sample, the user repeatedly squeezes and releases the wings 38 of the holder 12 to pump and/or extract blood from the finger 19 until a desired amount of blood is collected in the collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

Once a desired amount of blood is collected within the container 16, a blood collector portion including the collection container 16 can be detached from the collection device 10 in order to send a collected sample to a diagnostic instrument and/or testing device. The blood collector portion can be sealed via the cap or septum once removed from the collection device 10 to protectively seal the blood sample within the collection container 16.

### Multi-Size Capillary Blood Collection Kit

The previously described holder 12 and capillary blood collection device 10 provide advantages over conventional capillary blood collection devices. In particular, the holder 12 is configured to align with a patient's finger features, ensuring that the holder 12 consistently and securely remains in place and applies pressure in the correct location. This feature was accomplished by analyzing several sources of anatomical information (finger width and length, knuckle and artery locations) to limit squeezing to soft tissues near the collection site while avoiding pressure on hard tissues or blood vessels. Further, the wings 38 of the capillary blood collection device 10 are configured to apply pressure in two stages. The first stage has pressure on the finger 19 increased proportionally to the applied pressure. However, as intensity increases, the wings 38 begin to flex and bend until they touch and cannot displace any further. This two-stage application of pressure allows enough pressure to have adequate blood flow, but limits maximum pressure to avoid hemolysis.

In order to achieve such benefits in how pressure is applied to the finger 19 during the blood collection procedure, it is important that the holder 12 is sized correctly for the patient's finger, meaning that the holder 12 is neither too loose nor too tight to function properly. For example, if the holder 12 is too loose, the holder 12 may move relative to or fall off from the finger 19 during a blood draw procedure. If the holder 12 is too tight, the holder 12 may restrict blood flow or cause pain for the patient.

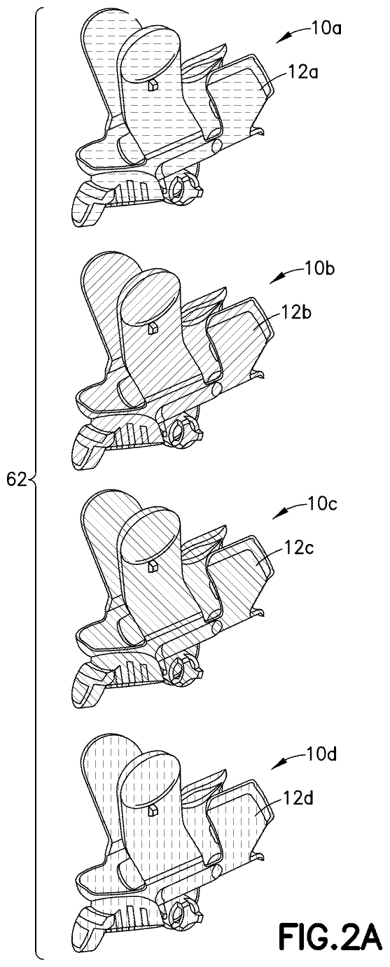

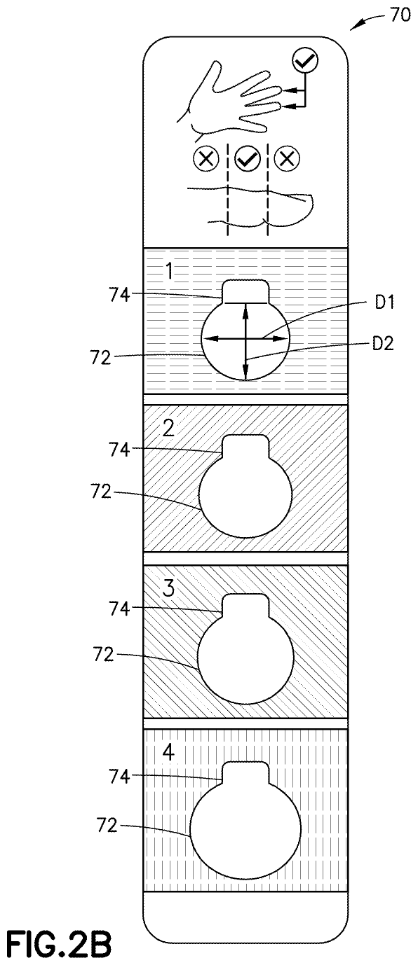

Since it is impractical to produce unlimited sizes of holders 12, a set or kit 62 including a discrete number of holders 12 of different sizes can be provided to a user. For examples, as shown in FIG. 2A, a set of capillary blood collection devices 10a, 10b, 10c, 10d, can include a device 10a with a small-sized holder 12a, a device 10b with a medium-sized holder 12b, a device 10c with a large-sized holder 12c, and a device 10d with an extra-large sized holder 12d. The different sized devices 10a, 10b, 10c, 10d are designed to fit patients with different sized hands and fingers. For example, the different sized devices 10a, 10b, 10c, 10d can be made to accommodate individuals with different finger widths, finger heights, and/or finger lengths (i.e., a distance between the fingertip and the second knuckle of the finger being used for the puncture procedure). In particular, sizes of different components of the devices 10a, 10b, 10c, 10d, such as a diameter of the first opening 22 and/or length of the finger receiving portion 20 of the holder 12 can be different for each size of device 10a, 10b, 10c, 10d to accommodate patients with different finger diameters.

Dimensions for the different sizes of the devices 10a, 10b, 10c, 10d can be determined, for example, from size charts and other anatomical data for average sized adult and/or pediatric patients. For example, the smallest device 10a can be sized to accommodate a finger width and finger length corresponding to 25^{th} percentile for an average adult person. Similarly, the medium device 10b can be sized for persons falling between the 25^{th} and 50^{th} percentiles for hand and/or finger size; the large device 10c can be sized for persons with a hand and/or finger size falling between the 50^{th} percentile and the 75^{th} percentile; and the extra-large device 10d can be sized for persons with a finger size falling between the 75^{th} percentile and the 100^{th} percentile. In some examples, size ranges for the devices 10a, 10b, 10c, 10d can be further optimized, for example, to minimize a mismatch between the holder size and a person's finger size for the largest possible number of individuals. In other examples, size ranges could be optimized so that a substantially equal number of persons within a population use each size of the device 10a, 10b, 10c, 10d. In that case, the size range for the small and extra-large devices (i.e., the difference between the maximum value of the size range and the minimum value of the size range) would be greater than for the medium device 10b and the large device 10c due to the substantially normal distribution of patient finger sizes. In order to determine an appropriate size of collection device 10a, 10b, 10c, 10d, a user may measure the patient's finger and/or perform some other sizing activity to determine the correct holder 12 size for a particular patient's finger that is being punctured.

The simulation device 110 of the present disclosure allows a user to practice determining which size capillary blood collection device 10a, 10b, 10c, 10d should be used for a particular finger and, once correct sizing is determined, to practice collecting a capillary blood sample using the correctly sized capillary blood collection device. With reference to FIG. 2B, a sizing tool or card 70 is illustrated that can be used for confirming sizing for a capillary blood collection device 10a, 10b, 10c, 10d. The sizing tool 70 can be, for example, a flat card including elliptical openings 72 sized to correspond to the different sizes of capillary blood collection devices 10a, 10b, 10c, 10d. The card 70 can be formed from laminated cardboard or from any other convenient material sufficiently strong to maintain its shape so that the card 70 can be disinfected between uses and reused multiple times. Alternatively, the sizing card 70 can be disposable, intended to be used one time for a particular patient prior to performing a blood draw procedure for the patient. In that case, the sizing card 70 can be formed from less substantial materials, such as thin cardboard or paper. In some examples, the openings 72 are elliptical openings 72 having a major diameter DI corresponding to a maximum finger width for the capillary blood collection devices 10a, 10b, 10c, 10d and a minor diameter D2 corresponding to a maximum finger height for the capillary blood collection devices 10a, 10b, 10c, 10d. The openings 72 can further include a notch 74 on a top portion of each opening 72. The purpose of the notch 74 is to make it easier for the user to insert the card 70 over the patient's finger and/or remove the card 70 from the patient's finger during sizing.

In some examples, a top opening 72 on the card 70 can have a major diameter DI and a minor diameter D2 corresponding to dimensions of the small device 10a. The bottom opening 72 can have a major diameter D1 and a minor diameter D2 corresponding to the extra-large device 10d. In order to use the sizing card 70, the user inserts the patient's finger to be used for the puncture procedure through the top opening 72 to see if the small device 10a should be used. For correct sizing, the finger should pass through the opening 72 up to or slightly past the first knuckle. If the top opening 72 is too tight, meaning that the opening 72 does not pass over the patient's finger to the first knuckle, then the small device 10a is too small for the finger. In that case, the user should test the finger using the other openings 72 of the card 70 (i.e., the openings 72 sized for the medium device 10b, the large device 10c, and, if needed, the extra-large device 10d), in order to determine which opening 72 is sized so that the opening 72 passes over the patient's finger to or slightly beyond the first knuckle. The user should use the size of capillary blood collection device 10a, 10b, 10c, 10d corresponding to the opening 72 that most closely fits the finger being used for the puncture procedure.

### Puncture and Blood Collection Simulation Device

Examples of the simulation device 110 that allow the user to practice using a capillary blood collection device, such as any of the capillary blood collection devices 10 shown in FIGS. 1A-1E, to collect a sample of capillary blood are shown in FIGS. 3-5 and 7A-7I. As previously described, the simulation device 110 allows the user, such as a phlebotomist, medical technician, nurse, physician, or another healthcare worker, to practice steps for using the capillary blood collection device 10 including, for example, sizing the collection device 10 (i.e., selecting a collection device 10 of an appropriate size for a patient's finger), inserting the collection device 10 over the patient's finger, activating the lancet 14 of the capillary blood collection device 10 to lance the finger, and collecting a capillary blood sample in a container 16 associated with the collection device 10. The simulation device 110 of the present disclosure can be used with any of the previously described capillary blood collection devices 10 or with any other collection device or collection assembly as are known in the art. The simulation device 110 can also be used with a wide variety of other medical devices configured to cut, puncture, damage, or otherwise manipulate areas of a patient's fingers, hand, wrist, forearm, or other appendages or extremity. For example, the simulation device 110 could be used to practice performing injections or blood draw procedures using syringes. In other examples, the simulation device 110 is used for practicing other procedures using scalpels and other surgical tools.

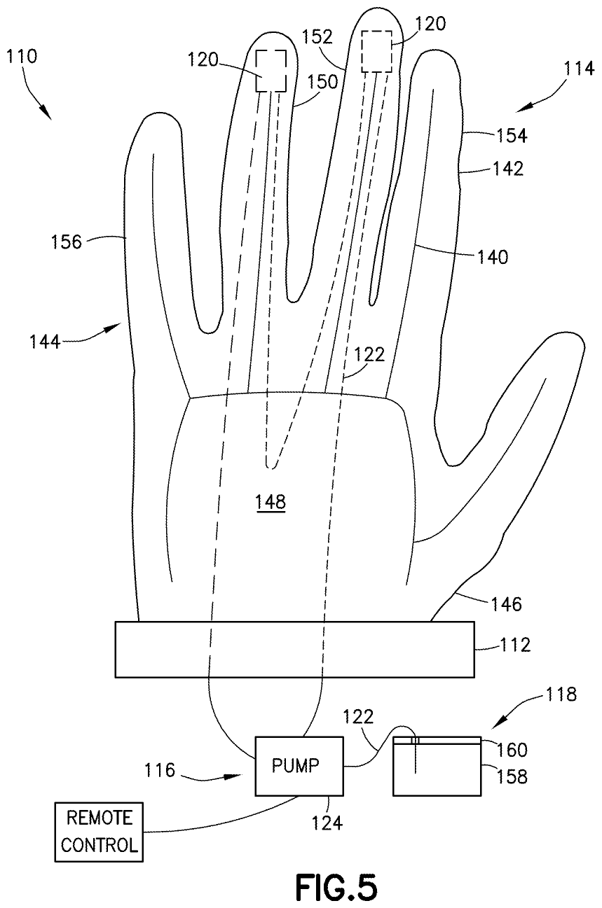

With reference to FIGS. 3-5, the simulation device 110 includes a base support 112, a puncture target 114 connected to the base support 112 configured to be repeatedly punctured by a lance of the capillary blood collection device 10 so that the device 110 can be used multiple times, and a fluid circulating system 116 for circulating a fluid (i.e., fake liquid blood) through the puncture target 114. As described in further detail in connection with FIG. 5, the fluid circulating system 116 can include a fluid reservoir 118, a puncture port 120 positioned in the puncture target 114 that can be punctured by the lance of the capillary blood collection device 10 so that fluid flows from the puncture site during the simulated blood collection procedure, tubing 122 extending at least partially through the target 114 between the reservoir 118 and the port 120, and a pump 124 for circulating fluid from the reservoir 118 to the port 120.

As described in further detail herein, the puncture target 114 is an appropriate size to be inserted into the holder 12 of the capillary blood collection device 10. When inserted in the holder 12, the puncture target 114, along with the port 120, can be punctured by the lance of the capillary blood collection device 10 in order to establish fluid communication between the container 16 of the capillary blood collection device 10 and the tubing 122 and reservoir 118 of the fluid circulation system 116. When the fluid communication with the tubing 122/reservoir 118 is established, the pump 124 can be activated to circulate fluid from the reservoir 118 through the tubing 122 to the port 120. The circulating fluid is expelled from the punctured port 120 and can be collected within the container 16 of the capillary blood collection device 10, which simulates a capillary blood collection procedure.

The base support 112 of the simulation device 110 can be any suitable supporting structure for maintaining the target 114 at an appropriate elevation and orientation to allow the user to practice performing the blood collection procedure. For example, the base support 112 can be configured to support the puncture target 114 with an appropriate vertical clearance distance (i.e., 10 inches, 8 inches, 6 inches, or 4 inches) between a lowest point or bottom portion of the target 114 and a surface (i.e., tabletop or counter) on which the base support 112 rests. In some examples, the base support 112 is a cabinet or housing including an interior space or compartment that houses components of the fluid circulation system 116, such as the pump 124, tubing 122, and/or reservoir 118.

As shown, for example, in FIG. 3, the base support 112 can include a substantially horizontal first member 126 that is configured to rest on a surface (i.e., the table or countertop). In some examples, the support member 126 can be a box or housing that contains components of the simulation device 110, such as the reservoir 118, pump 124, and/or tubing 122. The base support 112 can also include a substantially vertical plate or second member 128 extending from the first member 126. The puncture target 114 can be connected to and can extend from the vertical plate or second member 128, with a sufficient clearance distance (i.e., about 6 inches or more) between the target 114 and the first member 126 and/or the surface on which the simulation device 110 rests.

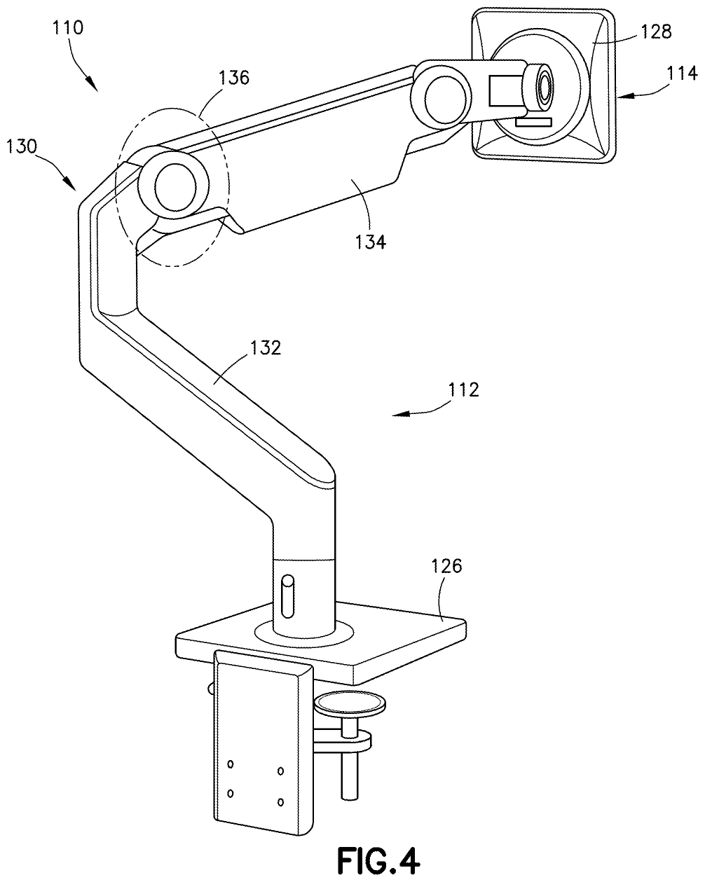

In some examples, as shown in FIG. 4, the base support 112 can include a bendable and/or flexible arm 130. The flexible arm 130 can be mounted between the first member 126 and the vertical plate or second member 128 to allow the user to adjust a position of the target 114. The flexible arm 130 can be similar to flexible support arms used with electronic displays, computer monitors, televisions, and similar devices. In some examples, the flexible arm 130 is rotatably mounted to the first member 126, allowing the user to rotate the arm 130 and target 114. The flexible arm 130 can include a first elongated member 132 and a second elongated member 134 connected by, for example, a hinge 136. By opening and closing the hinge 136, the user can adjust a height of the target 114 mimicking, for example, pronation and supination of a patient's wrist, which allows the user to practice moving the hand and to an appropriate height prior to performing the blood collection procedure.

In some examples, the base support 112 can include a heating device for warming the puncture target 114 so that the target 114 feels as lifelike and real as possible. For example, different types of electronic heating devices, such as heatable surfaces, coatings, or coils, can be embedded in or connected to the base support 112. In other examples, the base support 112 can be warmed by electronic devices positioned within the base support 112, such as the pump 124. Prior to use of the simulation device 110, the puncture target 114 can be moved in a downward direction towards the base support 112. The heating device can be turned on allowing the heating device to warm the puncture target 114. Once the puncture target 114 is warmed to a sufficient temperature, the user can turn off the heating device, raise the puncture target 114 to an appropriate (i.e., easily accessible) height, and can begin to perform the blood collection procedure.

With specific reference to FIGS. 3 and 5, the puncture target 114 is generally an elongated member of appropriate dimensions (i.e., an appropriate length and thickness) to be inserted into the holder 12 of the capillary blood collection device 10. The puncture target 114 can be formed from any material capable of being punctured multiple times by the lancet 14 of the capillary blood collection device 10 or by other blades without tearing or otherwise deforming. For example, the puncture target 114 can be formed from a support structure 140 covered by a sheet or cover 142 of a synthetic or natural elastomeric material. In some examples, the sheet or cover 142 can be self-sealing, meaning that the lancet 14 does not leave a noticeable opening, tear, or mark in the sheet or cover 142. In other examples, the material may not be self-sealing if, for example, the port 120 of the fluid circulation system 116 is capable of sealing shortly after being punctured by the lancet 14 of the capillary blood collection device 10.

In some examples, the puncture target 114 is an anatomically accurate model 144 of a human hand including, for example, a forearm, wrist 146, palm 148, four fingers (i.e., ring finger 150, middle finger 152, index finger 154, pinky finger 156), and thumb. In other examples, the puncture target 114 can be a more abstract model including, for example, cylindrical members sized to approximate a width, height, and length of a human finger. The hand model 144 can have a left-handed orientation, since most right-handed patients prefer to have punctures performed on their non-dominant hand. The wrist portion 146 or proximal portion of the hand model is engaged to the base support 112, with the palm 148 and fingers 150, 152, 154, 156 of the model 144 extending therefrom. The palm 148 of the hand model 144 is generally flat and faces downward, approximating appropriate positioning for the patient's hand during the puncture procedure. Also, the fingers 150, 152, 154, 156 can be spaced apart or separated so that the user can touch and manipulate individual fingers. In some examples, the fingers 150, 152, 154, 156 can be bendable, so that the user can move fingers 150, 152, 154, 156 that are not being punctured out of the way.

Since the capillary blood collection device 10 is generally used with either the middle finger 152 or the ring finger 150, the puncturable ports 120 of the fluid circulation system 116 can be disposed within distal portions of the ring finger 150 and/or the middle finger 152 of the anatomically correct hand model 144. Other fingers 154, 156 of the hand model 144 (which do not include puncture ports 120) can be bendable and/or capable of being bent away from the middle finger 152 and/or the ring finger 150 thereby allowing the user to easily access and manipulate the middle and/or ring fingers 150, 152 to practice the blood collection procedure.

The hand model 144 can be any size determined, for example, based on what types of blood draw procedures the user typically performs and/or wants to practice performing. In some instances, the hand model 144 can be sized in accordance with an average hand size for an average adult. For example, hand dimensions, such as finger length, finger circumference, palm circumference, and overall hand length, can be 50^{th} percentile or mean average dimensions for an adult. In other examples, such as for users that typically work with pediatric patients, the hand model dimensions can be for an average pediatric patient (i.e., 50^{th} percentile dimensions for a twelve year old patient).

In some examples, the support structure 140 of the hand model 144 is an internal frame structure having a configuration and appearance representing bones of the human hand. For example, the support structure 140 can include multiple elongated members that are fixedly or hingedly connected together to mimic connections between bones of the fingers and hand. The support structure 140 can be formed by any fabrication technique suitable for forming multiple connected members that are relatively small in size. For example, elongated members of the internal frame or support structure 140 can be made by 3D-printing using fabrication processes, as are known in the three dimensional printing art. The various elongated members of the frame or support structure 140 can be connected together by adhesives and/or mechanical fasteners, or in any other convenient manner. The cover 142 is positioned over the internal frame or support structure 140. The cover 142 can be made from an elastomeric material, such as natural or synthetic rubber, that has an appearance and feel of human skin. In other examples, the cover 142 can include leather or similar materials that have a similar look and feel to skin. Further, the cover 142 should be sufficiently thin to be punctured by the lancet 14 of the capillary blood collection device 10. The cover 142 can also have a color and/or texture that mimics the appearance of skin to provide a more realistic simulation experience for the user. The cover 142 can be connected to the internal frame or support structure 140 by any suitable connector, as are known in the art, including mechanical fasteners or connectors (i.e., staples, pins, threads, nails, or similar connectors) or using adhesives.

As previously described, in some examples, puncturable ports 120 of the fluid circulation system 116 are disposed in both the middle finger 152 and the ring finger 150 of the hand model 144. In that case, the ring finger 150 can be sized to receive one size capillary blood collection device 10 (i.e., the medium size capillary blood collection device 10b shown in FIG. 2A). The middle finger 152 can be sized to receive a larger size capillary blood collection device 10 (i.e., the large size capillary collection device 10c or the extra-large sized capillary blood collection device 10d shown in FIG. 2A). This configuration allows the user to practice sizing for the capillary blood collection device 10, as well as allows the user to practice performing blood draw procedures using different sized capillary blood collection devices 10.

With specific reference to FIG. 5, the fluid circulation system 116 of the simulation device 110 includes the fluid reservoir 118, the tubing 122 extending from the reservoir 118 through the puncture target 114, and the puncture ports 120 that can be located, for example, in the fingers 150, 152 of the target 114. The tubing 122 of the fluid circulation system 116 can be any flexible or rigid plastic tubing known in the art, such as tubing used for medical procedures (i.e., intravenous infusion procedures, dialysis procedures, and/or surgical procedures involving extracorporeal blood flow). The tubing 122 can also be flexible tubing commonly used as building materials, such as flexible tubing commonly used in HVAC systems. As previously described, the tubing 122 extends from the reservoir 118 to the puncture port 120. In some examples, the tubing 122 is entirely enclosed within the base support 112 and/or target 114. In other examples, some portions of the tubing 122 can be external to the base support 112 and/or target 114 and, for example, can be held in position against the base support 112 and/or target 114 by clips, fasteners, staples, or other connectors, as are known in the art.

The puncture port 120 of the fluid circulation system 116 refers to the portion of the fluid circulation system 116 that is positioned to be punctured by the lancet 14 of the capillary blood collection device 10. For example, as previously described, puncture ports 120 can be positioned near the distal end of the ring finger 150 and the middle finger 152 of the hand model 144. Desirably, the puncture port 120 is self-sealing, meaning that the port seals once fluid flow through the fluid circulation system 116 stops, so that the simulation device 110 can be used for multiple simulated blood collection procedures. In some examples, the puncture port 120 is simply a length of tubing connected to and/or integral with other segments of tubing 122 of the fluid circulation system 116. In other examples, the puncture port 120 can refer to a reinforced or more rigid section of tubing configured to be repeatedly punctured by the lancet 14 of the capillary blood collection device 10. Alternatively, the puncture port 120 can be an enlarged sealed cavity or recess in the finger(s) 150, 152 that is fluidly connected to the tubing 122 for providing fluid to and from the ports 120.

The pump 124 of the fluid circulation system 116 can be any of a variety of automatic or manually operated pumps, as are known in the art, capable of creating fluid flow through the fluid circulation system 116 at an appropriate flow rate. For example, the appropriate flow rate can be from about 2 μL/s to about 10 μL/s, or preferably, about 5 μL/s. In some examples, the pump 124 is an electric pump, such as a rotary pump, peristaltic pump, pneumatic pump, or other pump configurations known in the art. The pump 124 can be powered by a battery or by alternating electrical current supplied, for example, from a wall outlet. In other examples, the pump 124 can be a manually operated pump, such as a hand-operated piston pump, hand bulb pump, or similar device, as are known in the art, for forcing fluids through tubing.

In some examples, the pump 124 can be connected to a remote control 138 by wires or by a wireless connection, such as by a short range wireless data transmitter (i.e., a Bluethooth® transmitter). A user, such as the individual practicing use of the capillary blood collection device 10 and/or a trainer or proctor, can operate the pump 124 by, for example, turning the pump 124 on or off with the remote control 138 to control fluid flow through the fluid circulation system 116. In some examples, the user may also be able to control pump operating parameters, such as flow rate, using the remote control 138. In other examples, pump operating parameters can be adjusted with buttons, knobs, and other selectors on a housing of the pump 124. Beneficially, the remote control 138 allows another individual, such as the trainer, proctor, preceptor, or another person administering a test, to operate the pump 124 while remaining a distance away from the simulation device 110. This allows the trainer to observe the blood collection activities performed by the user and to actuate the pump 124 at appropriate times without distracting the user or otherwise hindering the user as he or she performs tasks associated with the simulated blood collection procedure.

The reservoir 118 of the fluid circulation system 116 can be any suitable container sized to hold a sufficient amount of liquid (i.e., fake blood liquid) to substantially fill the tubing 122 of the circulation system 116. In some examples, the reservoir 118 includes a separate and/or removable cup 158 (i.e., capable of being removed from the base support 112 and/or from other components of the fluid circulation system 116) with a lid 160 including openings for receiving ends of tubing 122 extending from the base support 112 or puncture target 114. In such instances, the user can set up the simulation device 110 by inserting the ends of the tubing 122 into the cup 158. When ready for use, the cup 158 can be inserted into a selected location inside the base support 112 or can remain external to the base support 112. After training exercises or testing with the simulation device 110 is completed for a period of time, the user can remove the lid 160 from the cup 158 and discard any remaining liquid. Following use, the user can fill the cup 158 with water or with a cleaning solution for circulating the water or cleaning solution through the tubing 122 and pump 124 of the fluid circulation system 116. In other examples, the reservoir 118 can be integral with the base support 112 and/or puncture target 114. In such cases, the user can fill the reservoir 118 with fluid by, for example, pouring fluid into the integral reservoir 118 through a fluid fill port.

### Method for Using the Simulation Device to Practice Performing Capillary Blood Collection

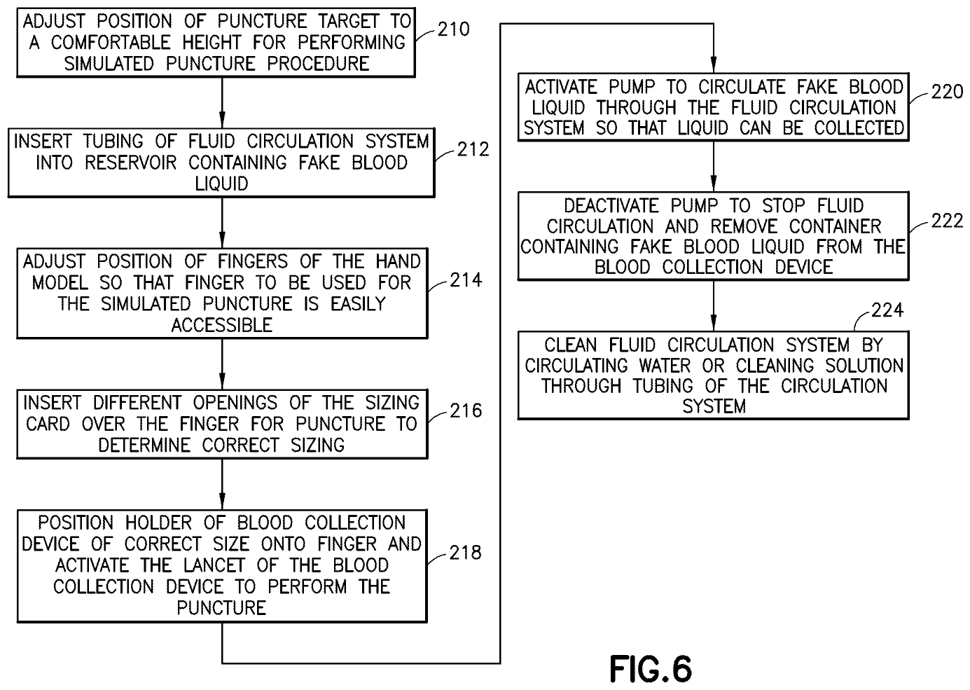

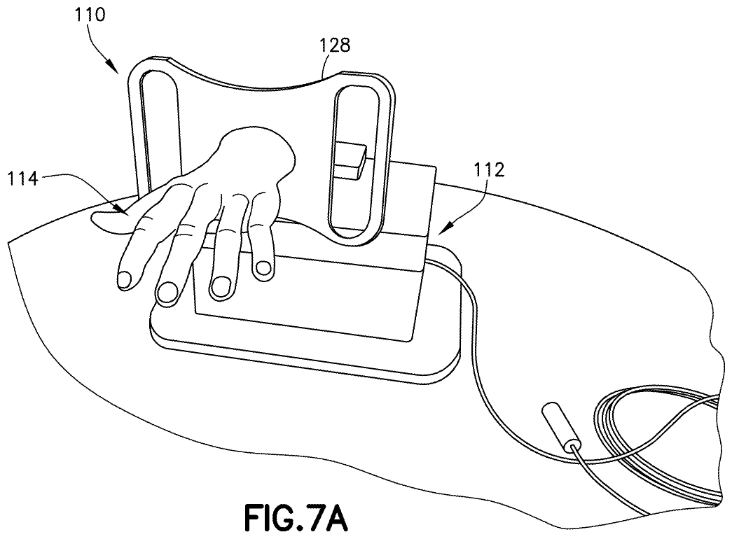

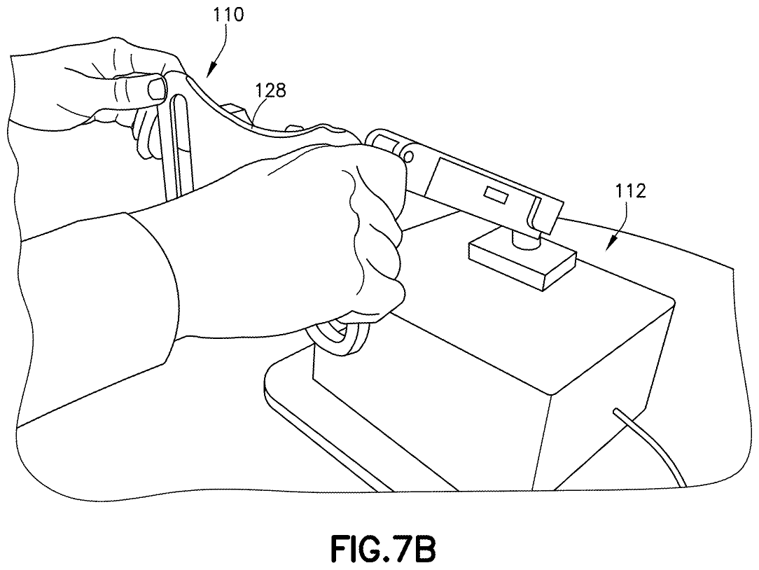

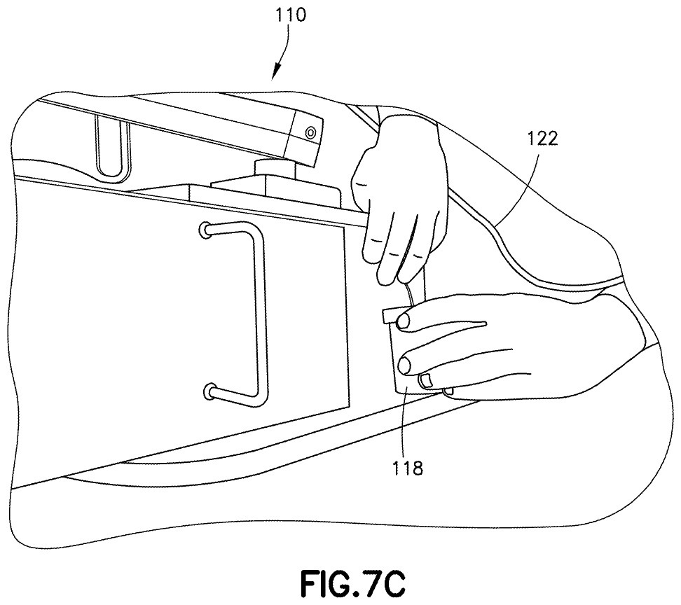

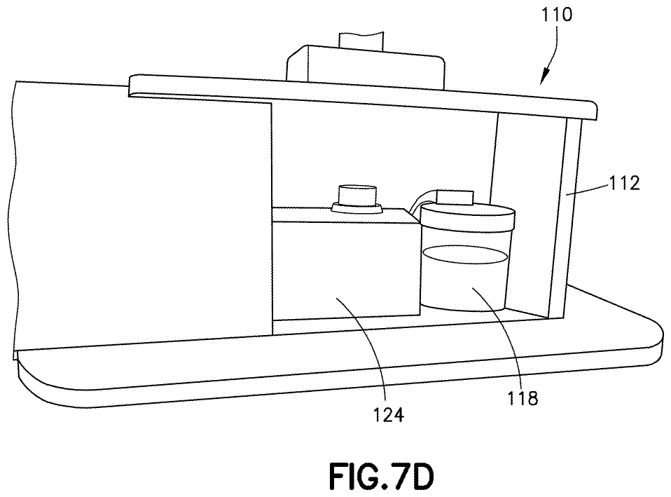

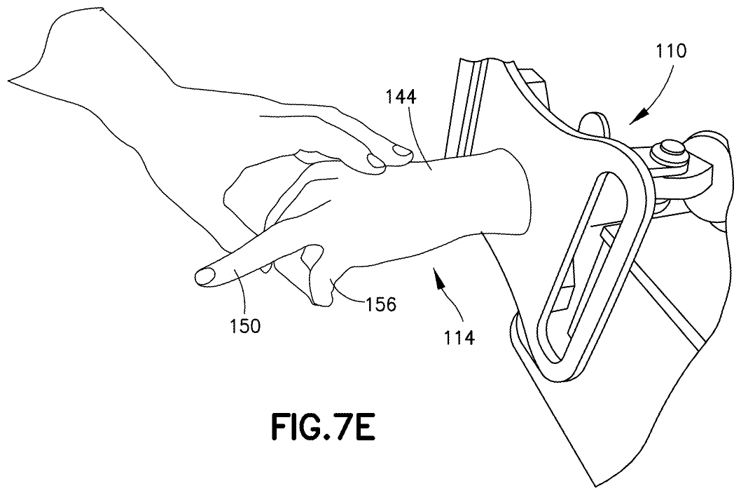

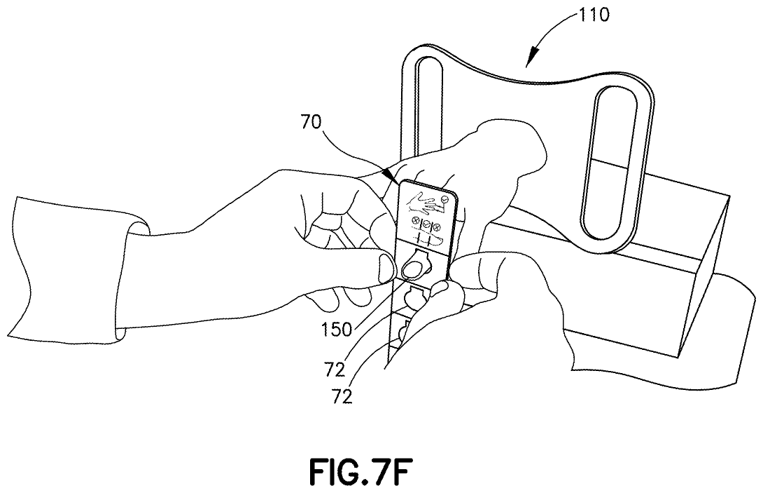

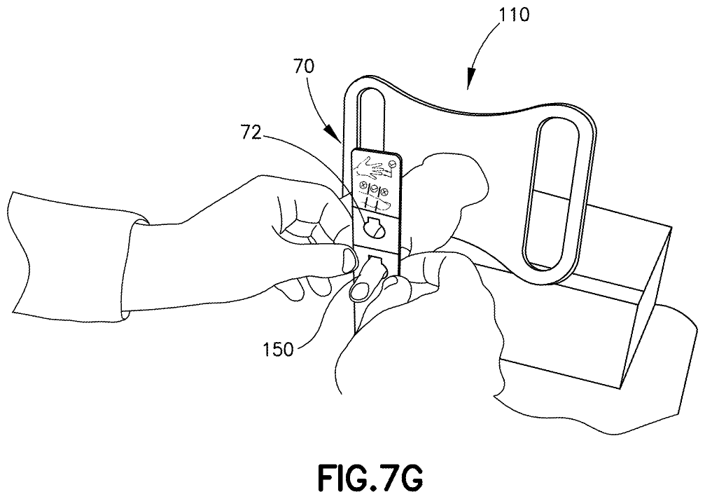

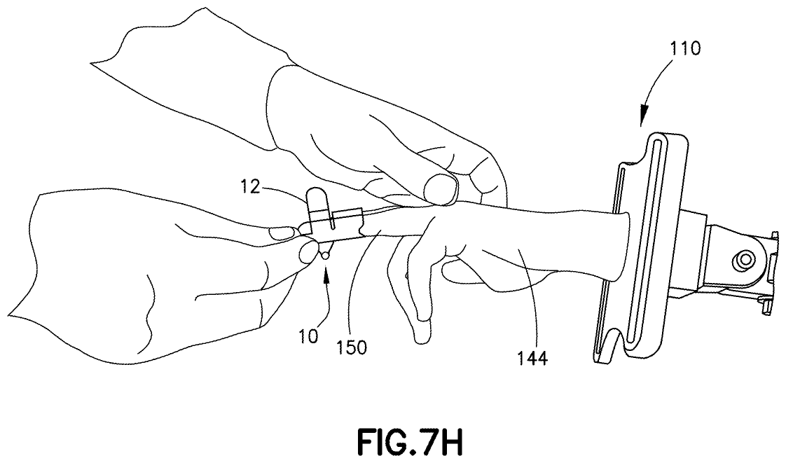

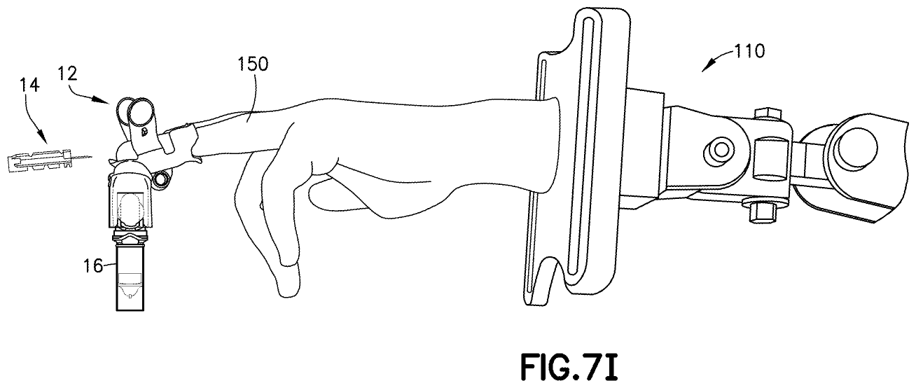

FIG. 6 is a flow chart showing a training method that can be performed by a user of the simulation device 110 to practice sizing and use of capillary blood collection device 10. As shown at step 210, the user first adjusts a position of the puncture target 114 or hand model 144 by, for example, grasping a portion of the vertical plate or second member 128 and/or target 114 and moving it up or down to a use position that is comfortable for the user. A drawing showing the user grasping the simulation device 110 to adjust a position of the puncture target 114 is shown in FIG. 7B. At step 212, the user next inserts a distal end of tubing 122 of the fluid circulation system 116 into the reservoir 118 to establish fluid communication between the reservoir 118, the pump 124, and the puncture port 120 of the fluid circulation system 116, as shown in FIG. 7C. Once the tubing 122 is inserted into the reservoir 118, the reservoir 118 can be inserted into an appropriate location within the housing of the base support 112, such as next to the pump 124, as shown in FIG. 7D. At step 214, once the fluid circulation system 116 is ready for use, the user next adjusts a position of fingers 152, 154, 156 of the hand model 144, so that the finger (i.e., the ring finger 150), which will be used for the puncture procedure, is easy to access and manipulate, as shown in FIG. 7E.

At step 216, the user next inserts the different openings 72 of the sizing card 70 over the finger 150 to determine what size capillary blood collection device 10 to use for the puncture and blood draw. As previously described, the openings in the sizing card 70 should be large enough to slide onto the finger 150 past the first knuckle. As shown in FIG. 7F, the top opening 72 on the sizing card 70 (corresponding to the small sized capillary blood collection device 10a) is too tight to slide over the finger 150 to the first knuckle. As shown in FIG. 7G, the second opening 72 (corresponding to the medium sized capillary blood collection device 10b) is the correct size for the ring finger 150.

At step 218, the user next inserts the holder 12 of the capillary blood collection device 10 of the correct size onto the ring finger 150 of the hand model 144, as shown in FIG. 7H. For semi-integrated capillary blood collection devices (i.e., capillary blood collection devices 10 where the lancet 14 and/or container 16 are not integrally formed with the holder 12), the user may also attach the lancet 14 and container 16 onto the holder 12, after the holder 12 is attached to the finger 152 of the hand model 144. Once the holder 12 and/or other components of the capillary blood collection device 10 are connected to the ring finger 150 of the hand model 144, the user activates the capillary blood collection device 10 to puncture the finger 150 as previously described in connection with FIGS. 1A-1E. Specifically, once activated, the lancet 14 of the capillary blood collection device 10 punctures the cover 142 of the hand model 144 and the port 120 of the fluid circulation system 116 to establish fluid communication between the container 16 of the capillary blood collection device 10 and the components of the fluid circulation system 116.

At step 220, shortly before or just after the puncture is performed, the user (or the trainer or proctor) can activate the pump 124 to circulate the fluid (i.e., fake blood liquid) through the fluid circulation system 116 of the simulation device 110. The circulating liquid is expelled through the punctured port 120 and into the container 16 of the capillary blood collection device 10, thereby simulating a capillary blood draw, as shown in FIG. 7I. As previously described, as the liquid is being collected, the user may manipulate the wings 38 of the capillary blood collection device 10 to enhance fluid flow, which effectively pumps the circulating liquid from the tubing 122 and/or puncture port 120 into the collection container 16 of the capillary blood collection device 10.

At step 222, once an appropriate amount of the fluid is collected in the container 16, the user can turn off the pump 124 to stop fluid from circulating through the fluid circulation system 116 and remove the container 16 of the capillary blood collection device 10 from the holder 12, thereby completing the blood draw procedure. The user may also remove the holder 12 from the ring finger 150 of the hand model 144. As previously described, the puncture port 120 of the fluid circulation system 116 is intended to be self-sealing, such that once the holder 12 and lancet 14 are removed, the port 120 seals allowing the simulation device 110 to be used for additional simulated blood draw procedures.

At step 224, if no other simulated procedures are to be performed for a period of time using the simulation device 110, the user can clean the fluid circulation system 116 of the simulation device 110 by discarding any remaining liquid from the reservoir 118. Once the liquid is discarded, the user can fill the reservoir 118 with water or a cleaning solution and activate the pump 124 to circulate the water or cleaning solution through the tubing 122 of the fluid circulation system 116.

While different examples of the simulation device and method are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A simulation device allowing a user to practice use of a capillary blood collection device, the simulation device comprising:
a base support;
a puncture target connected to the base support configured to be repeatedly punctured by a lancet of the capillary blood collection device to practice use of the capillary blood collection device; and
a fluid circulating system comprising a fluid reservoir, at least one port positioned within the puncture target, tubing extending at least partially through the puncture target between the reservoir and the at least one port, and a pump for circulating fluid from the reservoir to the at least one port,
wherein the puncture target is sized to be connected to the capillary blood collection device so that the capillary blood collection device punctures the target and the at least one port to establish fluid communication with the tubing and reservoir, and
wherein, when the fluid communication is established and the pump is circulating the fluid from the reservoir, the fluid is expelled from the at least one port and is collected within a container of the capillary blood collection device.

2. The simulation device of claim 1, wherein the base support is configured to support the puncture target with at least 6.0 inches of vertical clearance between the puncture target and a bottom of the base support.

3. The simulation device of claim 1, wherein the base support comprises a first member configured to rest on a surface and a second member extending from the first member, wherein the puncture target extends from the second member of the base support.

4. The simulation device of claim 1, wherein the base support comprises a flexible arm rotatably and/or pivotally mounted to a support member.

5. The simulation device of claim 4, wherein the flexible arm comprises at least one hinge configured to bend in one or multiple directions to adjust a position of the puncture target relative to the support member.

6. The simulation device of claim 4, wherein the flexible arm pivots and/or rotates to raise or lower a height of the puncture target relative to a bottom of the base support.

7. The simulation device of claim 1, wherein the pump comprises an electric pump.

8. The simulation device of claim 1, wherein the pump comprises a manually operated mechanical pump.

9. The simulation device of claim 1, wherein the fluid circulating system further comprises a remote control operatively connected to the pump configured to operate and/or control a flow rate of the pump.

10. The simulation device of claim 1, wherein the pump draws fluid from the reservoir at a flow rate of about 5 μL/s.

11. The simulation device of claim 1, wherein the fluid in the reservoir comprises fake blood liquid.

12. The simulation device of claim 1, wherein the puncture target comprises a substantially anatomically accurate hand model comprising a proximal end portion connected to the base support and a distal end portion comprising a plurality of elongated members representing fingers, and wherein the fluid circulating system comprises a plurality of ports positioned in the elongated members representing the fingers.

13. The simulation device of claim 12, wherein dimensions of the hand model are selected based on 50^{th} percentile anatomical measurements for hand size for an adult.

14. The simulation device of claim 12, wherein the plurality of elongated members of the hand model comprise at least a ring finger member sized to receive a first size of the capillary blood collection device and a middle finger member sized to receive a second size of the capillary blood collection device that is larger than the first size.

15. The simulation device of claim 12, wherein the hand model is oriented in a left-hand orientation, and wherein the hand model is positioned with a flat downwardly facing palm and with the plurality elongated member representing fingers spaced apart.

16. The simulation device of claim 12, wherein the hand model comprises an internal frame that mimics bones of a human hand and an elastomeric cover over the internal frame configured to mimic a look and feel of human skin.

17. The simulation device of claim 16, wherein the cover comprises an elastomeric sheet.

18. The simulation device of claim 12, wherein a first port of the plurality of ports is positioned in an elongated member of the plurality of elongated members that represents a middle finger, and a second port of the plurality of ports is positioned in an elongated member representative of a ring finger.

19. The simulation device of claim 18, wherein elongated members representative of fingers other than the middle finger or the ring finger are bendable, capable of being bent away from the middle finger and/or the ring finger thereby allowing the user to access and manipulate the middle and/or ring fingers to practice a procedure.

20. A method for practicing sizing and use of a capillary blood collection device, the method comprising:
inserting an elongated member of the puncture target of the simulation device of claim 1 through one or more openings in a sizing card to identify an opening that best fits the elongated member of the puncture target;
inserting a holder of the capillary blood collection device of a size corresponding to the identified openings onto the elongated member of the puncture target;
activating the capillary blood collection device to puncture the elongated member and at least one puncture portion of the capillary blood collection device; and
activating the pump of the fluid circulation system, thereby allowed circulating fluid to collect in a container of the capillary blood collection device.
